(19) United States
(12) Patent Application Publication
(10) Pub. No.: US 2025/0050174 A1
(43) Pub. Date: Feb. 13, 2025

(54) OMNI TORQUE TRAINING

(71) Applicant: Christian Malcolm, Thousand Oaks, CA (US)

(72) Inventor: Christian Malcolm, Thousand Oaks, CA (US)

(21) Appl. No.: 18/754,658

(22) Filed: Jun. 26, 2024

Related U.S. Application Data
(63) Continuation of application No. 17/889,269, filed on Aug. 16, 2022, now abandoned.
(60) Provisional application No. 63/234,342, filed on Aug. 18, 2021.

(51) Int. Cl. A63B 24/00; A63B 21/00

(52) U.S. Cl. CPC A63B 24/0087; A63B 21/151; A63B 21/4001; A63B 24/0075; A63B 2024/0093; A63B 2220/806

(57) ABSTRACT

Disclosed are example embodiments of systems and methods for exercise including an exercise machine including a multiple directional force component configured to apply a multiple directional force to an exercise machine-human body interface, the multiple directional force component further configured to provide a divergent intensity to the exercise machine-human body interface to provide the human body with a resistance during a physical exercise and an artificial intelligence (AI) component configured to control the multiple directional force component. In an example, the AI component may control the multiple directional force component based on morphological characteristics and variability of running speed parameters including at least one of stride length, stride frequency, explosive drive force, and arm drive.

### CROSS-REFERENCE TO RELATED APPLICATIONS

The present application is a continuation of U.S. patent application Ser. No. 17/889,269, filed Aug. 16, 2022, which claims priority to U.S. Provisional Patent Application No. 63/234,342, filed Aug. 18, 2021, which are hereby expressly incorporated by reference herein.

### TECHNICAL FIELD

The disclosure relates generally to the field of physical fitness, specifically and not by way of limitation, some embodiments are related to exercise.

### BACKGROUND

Strength training, resistance training, cardio weight training, balance and flexibility workouts, sports specific training, endurance training, and weightlifting are all important parts of any exercise routine for the development and performance of the elite human body. These training techniques and exercises have been predominately created as either linear or lateral movements with a singular point of intensity or tension on each movement. An example of a linear exercise with a single point of intensity is a single arm bicep curl using a dumbbell, a very common and popular exercise. Traditional training programs require multiple exercises with multiple angular motions and rotations to target and achieve the desired muscle activation, development, and performance. An example of a typical bicep workout routine using a singular directional intensity may include the following example exercises: Standing Barbell Curl, Bent Bar Preacher Curl Wide, Straight Bar Preacher Curl Close, Hammer Curl, Incline Dumbbell Curl, Reverse-Grip Curl, Cable Curl, Seated One Arm Concentration Curl.

In addition to the number of exercises, machine platforms, bars, benches, etc. needed to reach and target the desired muscular output across the specific muscle group, heavier and heavier weight must often be applied to the same exercises to achieve muscular gains and build strength over time. Increasing weight on exercises can and often does induce or cause injury to connective tissue (ligaments and tendons) and joints. This ever-increasing weight can potentially lead to complete muscle tears, torn connective tissue, and joint deterioration over time. Also, weight increment limitations of dumbbells may typically be 5 pounds, 10 pounds, 20 pounds, 25 pounds, 30 pounds, 35 pounds, 40 pounds, 45 pounds, etc. and 5 pounds, 10 pounds, 25 pounds, 35 pounds, and 45 pounds for weighted plates. The use of 5-pound increments rather than more granularity may increase the chance of injury. For example, this jump of incremental weight often outpaces muscular growth and performance.

Time is another factor that may increase potential injury. Increasing workout time and repetitions are a growing concern for potential injury, especially in athletes that perform repetitive sports specific movements. Some exercise enthusiasts and elite athletes may desire a more effective way to activate muscle groups and achieve a high level of performance without risking injury due to excessive weight, increased repetitions, and numerous exercises. A new system of training is needed to increase muscle activation and limit potential injury. Multiple directional activation of muscle groups and connective tissues at one time provides a step forward in training the human body to its elite potential with less injury and stress on the body.

### SUMMARY

Disclosed are example embodiments of systems and methods for exercise including an exercise machine including a multiple directional force component configured to apply a multiple directional force to an exercise machine-human body interface, the multiple directional force component further configured to provide a divergent intensity to the exercise machine-human body interface to provide the human body with a resistance during a physical exercise and an artificial intelligence (AI) component configured to control the multiple directional force component.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating an example exercise machine in accordance with the systems and methods described herein.

FIG. 2 is a diagram illustrating a frontal view of the example exercise machine of FIG. 1

FIG. 3 is a diagram illustrating a side view of the example exercise machine of FIG. 1

FIG. 4 is a diagram illustrating a side view of a bench accessory that aids in creating additional exercises that require back support or body positioning that cannot be achieved from a standing position.

FIG. 5 is a diagram illustrating a side view of a treadmill accessory that aids in creating cardio-based exercises when coupled with the machine.

FIG. 6 is a diagram illustrating four possible accessories that link the human body to the invention: 1) triceps bar, 2) multiple directional handles, 3) long bar, and 4) swivel handles.

FIG. 7 is a flow diagram of an example method in accordance with the systems and methods described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of example exercise systems will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using mechanical components, electronic hardware, computer software, or any combination thereof. Whether some of these elements are implemented as hardware or software depends upon the particular application and design constraints imposed on the overall system.

By way of example, an element, or any portion of an element, or any combination of elements may be implemented as a “processing system” that includes one or more processors. For example, an artificial intelligence (AI) component may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an, electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

An example new method of training is possible by applying directional intensity to the body at safe points of flexion, extension, abduction, circumduction and rotation. For example, applying a large amount of static weight on a singular point of flexion at a joint may create undue stress when creating momentum; however, applying increasing or gradual intensity on the body from multiple directions or angles after the initial flexion joint is in motion may be a safer and more effective way to active muscle groups without stress. An example may utilize a latex band for exercise where the intensity increases as the band is expanded during the motion and therefore the latex band applies the greatest intensity to the body at the end of the flexion point where stress on the joint may not be as dangerous. A plurality of bands or cables with directional intensity applied to the body as momentum builds with motion, would offer the greatest muscular activation without added stress to joints and connective tissues. Perhaps with the exception of powerlifting there is generally no sport that may benefit from overloading the body with weight when considering risk versus reward and overall performance versus injury.

Variable intensity may include, but is not limited to, physical weights, weighted cables, magnetic disc tension, pulley motors, flywheel systems and cables, intensifying band tension, assistance or resistance band tension, continuous variable wave or pulsing forces on the body, torque tension created by varying angles, and any combination thereof. Multiple directional intensity, e.g., omni training, may transfer resistance to the body through various accessories: handles, bars, gauntlets, straps, belts, vests, and any accessory that has multiple attachment zones linking the human body to the machine or system. These accessories may also be modular in nature so that a single bar is not necessary for each movement and configuring multiple bars and handles with variable intensity zones may be constructed from a few designed parts. In addition, adjustable benches, a stationary bicycle, a flat treadmill, and a piece of exercise equipment may be placed inside or near the machine to utilize directional intensity training while exercising, walking, running, riding, or performing other movements. An example of this would be a stationary bicycle that is placed in the center of the machine with the handles positioned close to the arms of the user so upper body exercises may be performed while riding a stationary bicycle.

Using these forms of weights or tension variables coupled with multiple directional adjustable intensity may be an effective way to produce muscular activation and increase performance levels while reducing stress or strain to connective tissue and joints. The technique of building muscle, increasing activation, and core engagement, may strengthen the overall muscular skeleton simultaneously, thus creating a stronger frame to withstand future performance growth and reduce potential injuries.

Equally as important as using multiple directional force coupled with divergent intensity on the body is the implementation of an artificial intelligence trainer combined with real-time performance feedback. The ability to give proper virtual instruction and identify the effectiveness of the exercises based on identifying muscle soreness, recovery time, and fatigue level by a user and user groups may help the user or user groups achieve success. Considering that each user's body is different but also similar in many ways, assisting and altering user workout plans, exercises, divergent intensity, and directional force applied to the movements may improve individual and user group performance and may prevent future injury across the entire user platform.

An example component to the artificial intelligence trainer is a virtual body scan. Each user can opt into an electronic body scan performed by the machine's cameras to create a virtual user avatar depicting the user's unique muscular skeleton, as well as additional fitness data, such as body fat, injuries, bone mass, height, reach, as well as other fitness data. The user's avatar may be manipulated to identify performance feedback and a muscular fatigue rating after workouts over time (day 1 recovery, day 2 recovery, day 3 recovery, for example).

The initial assessment of the user's 3D body composition may be identified and created by tracking and recording performance of exercises on the machine from multiple data points and machine cameras. Data collected by using different intensities and directional forces from exercises may aid in the construction of each user's muscular skeleton. The virtual 3D computer model may include but is not limited to strength, endurance, muscular activation, muscular elasticity, and estimated muscle size. The user's muscular skeleton avatar may be displayed on the display screen or may also be shown in a virtual reality headset to help assist the user during training and while performing recovery feedback.

Understanding how each user's body reacts and performs under the various exercise routines may be used to evaluate the effectiveness of the various programs offered and may alter future workouts to maximize efficiency with less potential injury over time. The virtual 3D muscular assessment may be continually updated based on machine learning and user feedback of performance, muscular fatigue and recovery time from the mobile application or interface. Over time the user avatar may become more specialized and accurate. User feedback may identify if the exercise routines are targeting the intended muscular activity and growth, as well as, impacting actual physical performance of the athlete. This also provides a social interaction component that users can share their positive and negative experiences across the entire platform, producing opportunities for the workout creation team to engage with users.

An example of how the user might enter recovery information into the mobile application or directly on the machine with or without an assisted virtual reality headset is as follows: Step 1, user is alerted in the morning shortly after sleep to complete a series of stretches (either on or off the machine) related to the previous day's workout routine. Step 2, user may identify on their own 3D skeleton model where they feel soreness, tightness, or pain. Step 3, user may rank their feedback from a scale of 1-10 identifying the intensity of the muscle soreness or additional recovery time needed (intensity soreness scale will be identified to help align feedback without user bias or variances). Based on the user's feedback the feedback system or application may alter or delay the next workout or offer additional stretching techniques to improve blood flow, massage techniques, and other methods of stimulating recovery. Data may also be shared with the user so the information can be easily transferred to a physical therapist or chiropractor for assistance in recovery.

Once user feedback is completed, the mobile application or interface will upload the information to the machine learning, e.g., Artificial Intelligence (AI) system, “to the cloud” under their unique and protected profile to assess workout routine effectivity and offer modified or increased workouts in the future based on performance. The AI system may also identify for the user potential areas of growth, calories burned, stress level, etc. and what to expect during the next custom workout. This constant interaction will improve user engagement and keep the user active and motivated to stay with the program. The system may also interface with wearable technology (smart watch or band) monitoring the user's sleep, resting pulse, heart rate, water intake, diet and nutrition to better optimize training and performance. The system will be able to perform stress tests on the body and compile an overall health assessment with regards to cardiovascular health, stress, muscles and joints, and other reports based on the wearable trackable health data.

A user may also provide feedback data generated to benefit the larger user community. Feedback utilization may include the collecting of data to create new workout routines and or modify programs best suited for the user's unique goals and continued performance, as well as user groups and subgroups. The more active users and groups that participate in the machine learning feedback utilization program the more effective the programs become over time, benefiting all users and sub-group profile types with exact training methods, recovery time, and injury prevention. For example, if an exercise routine with a specific ratio of repetitions to directional weight intensity results in a more positive user experience across a specific user demographic, then the exercise routine may be maximized across potential candidates that match the user demographic. Maximizing the exercise routine across potential candidates that match the user demographic may improve the user experience and user outcome.

The 3D avatar feedback system may also be utilized as a tool for injury prevention by identifying pain associated with specific movements and various exercise routines on joints, ligaments, and tendons. Continuous feedback is critical for the machine learning process to offer the best workouts possible for each user's specific goals and performance level with less potential injury. For example, a user (user one) with “tennis elbow,” lateral epicondylitis, may identify an exercise or exercises that aggravate the condition; another user (user two) also with lateral epicondylitis identifies a similar exercise that targets the same muscle group but varies slightly by angle or intensity which did not aggravate user two's condition. Therefore, modifying user one's exercise to match user two's exercise may offer user one a modified workout with respect to a lateral epicondylitis condition. Over time the system's understanding of each user's body will greatly improve or modify workouts resulting in better performance, muscular growth, balance, form and technique, and muscular skeleton of the user especially those with injuries. A more specialized feedback system may also be created to help with physical therapy exercises and training for people with physical disabilities resulting from disease, accident, or catastrophic injury.

The machine learning may understand how the exercise is performed by each specific user and store user history or memory to include: degree angle from joint, intensity angle, weight applied, tension by directional intensity, force, speed, time, and overall impact of user's biometrics on the performance of the exercise, (tracking physiological characteristics which are related to the shape of the body including, but not limited to, height, weight, strength, muscle mass, reach, age, gender, or other physiological characteristics). Therefore, even if user one is not biometrically like user two, the machine may be able to adjust and modify all system variables to best mimic the exercise from one user to another regardless of physical stature and strength of each user. The machine learning algorithm may offer the best modified exercises to all user groups until the user feedback reaches beneficial status. Each exercise can also be identified by system benefit status, such as New Workout, System Learning, or Final. Audio commands or cues may also assist the user's performance as a personal trainer would instruct a client. The level of cues may vary by user preference and overall performance level. User's that need more instruction can set the Cue Command to high, where advanced users may set the virtual trainer mode to low or less interactive. Cue Commands may also be generated in many languages and accents that are beneficial to the user and create a fun and engaging environment.

Promoting healthy muscle development and proper muscle memory with less potential injuries may provide continued user interaction and success. Promoting healthy muscle development and proper muscle memory may be particularly advantageous for all levels of athletic performance from amateur to elite professional athletes, where performing sports specific movements with directional intensity is essential to building proper muscle memory that best mimics real performance and execution. Feedback on muscle fatigue, including game or sports performance, may also be performed, and may become a valuable data point that can be built on top of a 3D Avatar, muscular skeleton model to benefit users.

Specialized programs may also be developed for specific athletes or sports and monitored by a coach or trainer for performance feedback and completion of routines and workout programs for accountability. An example of this is a high school track athlete who enters specific data such as 100-meter, 200-meter, and 400-meter results and the AI system creates a custom program to improve speed, and performance time based on morphological characteristics and variability of running speed parameters (stride length, stride frequency, explosive drive force, and arm drive). Over time the athlete will be able to track performance and growth of sports specific muscle groups to achieve better results in competition.

The Artificial Intelligence of the machine may also be coupled with Virtual Reality to mimic sports specific movements against appointments or for individual competition. An example of this would be offensive linemen drills where the user/athlete is mimicking explosive leg drive and arm drive with elbows inside and hands up. Or users can participate in a premium virtual reality experience and train with their favorite celebrity trainers or celebrity fitness personalities like Dwayne Johnson for a daily drop-in rate or a monthly subscription that can be promoted throughout the platform and across social media.

In addition to the user feedback utilization program based on the workouts, or Virtual Reality training options other additional components such as supplements, vitamins, and diet programs may also be offered, tested, and effectiveness examined to maximize user performance and experience. This can be particularly advantageous when comparing performance results and recovery on populations that have implemented a diet and supplement program versus those who have not.

A detailed description of the invention and one or more of its embodiments is provided below with figures that illustrate the principles of the invention. The invention is described but not limited to any one embodiment, as the scope of the invention comprises numerous principles and practices. Specific details are set forth in the description to provide a framework only and do not limit the invention by the examples and illustrations. The invention may be practiced according to the claims without some or all these specific details.

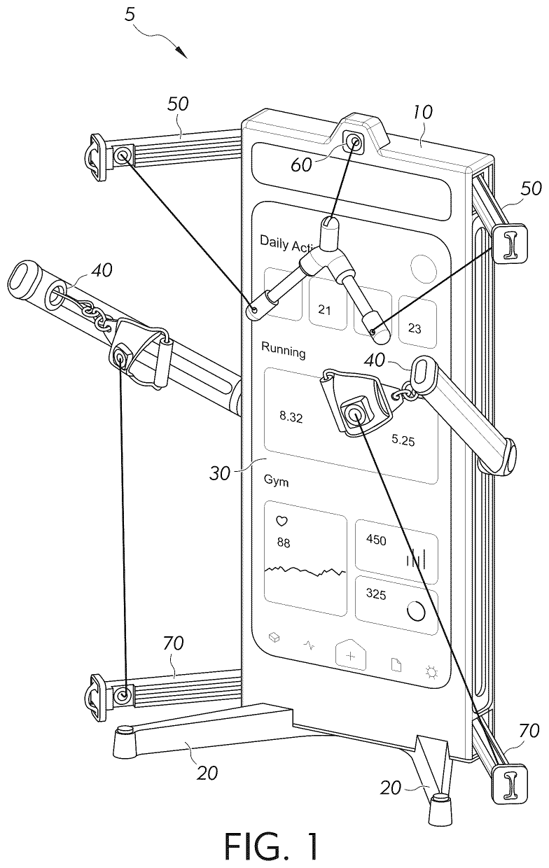

FIG. 1 is a diagram illustrating an example exercise machine (5) in accordance with the systems and methods described herein. The example exercise machine (5) includes a master housing unit (10) that may protect and support the electronics and internal workings of the machine (5). The example exercise machine (5) includes a support stand (20) that may provide stability and support, e.g., strong enough to support high intensity exercises. The example exercise machine (5) includes a user screen (30) that may depict a user's profile, physical activity monitoring (e.g., heart rate, calories burned, duration of the workout, countdown, or other data), an exercise routine by repetition either programed or live instruction, and any other user requested information. The example exercise machine (5) includes a set of main arms (40) that may be capable of 4-axis rotations on a joint and vertical track with numerous points of directional intensity including an internal arm track that allows the cable eye or tension band to travel horizontally within the arm. The example exercise machine (5) includes a set of top (right and left) cable track housings (50) that hinge on a joint close to the master housing unit (10) on a horizontal axis and well as horizontal on a track inside the arm housing providing multiple attachment zones for cross directional intensity. The example exercise machine (5) includes a center cable eye (60) providing directional intensity from the center top of the machine. The example exercise machine (5) includes a set of bottom (right and left) cable track housings (70) that hinge on a joint close to the master housing unit (10) on a horizontal axis and well as slide up and down a track inside the housing providing multiple attachment zones for cross directional intensity.

In total the drawing describes 7 unique attachment zones that may create an effective system of multiple directional intensity on body during physical activity, sports movements, and therapeutic motion. The drawing is one example embodiment and other embodiments may not be limited to only 7 zones. For example, additional zones may be added, or zones may be eliminated and replaced by coupling straps that may link multiple cables together to create one zone with multiple directional forces, not pictured.

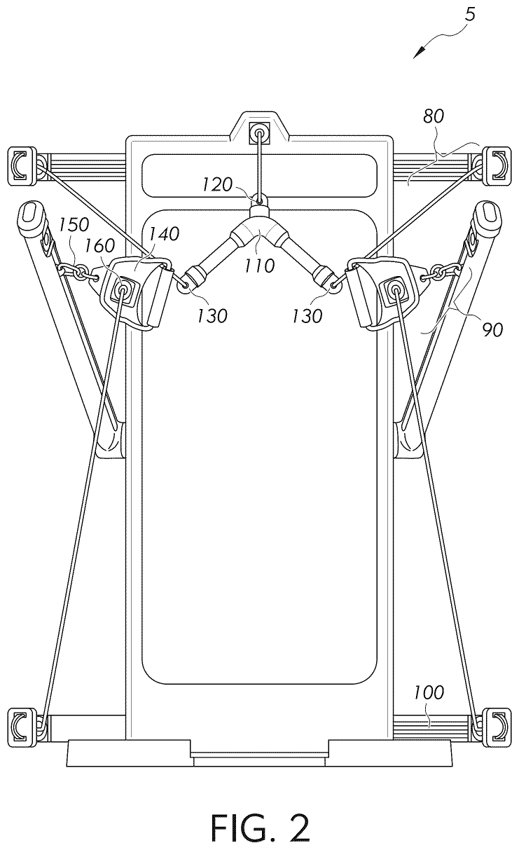

FIG. 2 is a diagram illustrating a frontal view of the example exercise machine (5) of FIG. 1 illustrating an example featuring several zone attachment accessories, cables, and how they might be linked together by the system for applying multiple directional intensity on the body during physical exercise. The machine setup includes adjustable arms protruding from the housing unit on the top, middle and bottom with internal slide tracks, top (80), middle (90), and bottom (100). The machine setup includes, at the top of the machine, a triceps bar accessory (110) with 3 potential attachment zones, top (120) and two on each side of the bar at its base (130). The machine setup includes a second accessory feature that may be the multiple directional handles (140) that have two unique attachment zones, one at the base of the handle (150) and one located in the center of the hood (160).

These unique zone attachment accessories offer multiple intensity directional training across a wide range of exercises. Each accessory provides numerous setup angles, heights, distances, depths, and range of motion to accommodate all user physical demographic profiles including height, reach, flexibility, range of motion, and injuries. Altering the angle of the exercises, directional intensity, range of motion, and weight can target and activate the muscular skeleton of the user to better achieve their unique goals and performance over time.

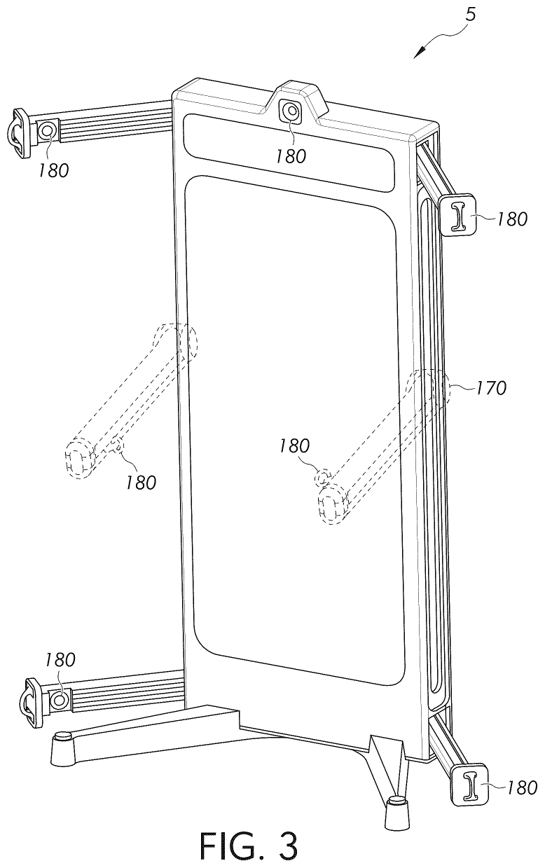

FIG. 3 is a diagram illustrating a side view of the example exercise machine (5) of FIG. 1 highlighting the main side joint of the middle arm (170) and the seven cables (180). Each cable may be pulled into alignment from a resting or starting position, attached to an accessory (bar, glove, gauntlet, belt, vest, strap, etc.), and adjusted or positioned into the correct alignment prior to the activation of the intensity. This allows the user to configure the setup without intensity or weight. The user may perform the exercise with proper range of motion and technique (instructed by the machine) without intensity to become acclimated to the movement without tension. After the user is confident in performing the desired exercise, they may activate the intensity by a verbal command or by touching the screen to activate. In addition, a timer or countdown may begin to alert the user when the intensity activation on the accessory will begin. Furthermore, after the exercise is completed and the number of desired repetitions performed the machine may deactivate the intensity gradually to allow the user to reduce stress on the body while securing the accessory or cables back into the starting or resting position.

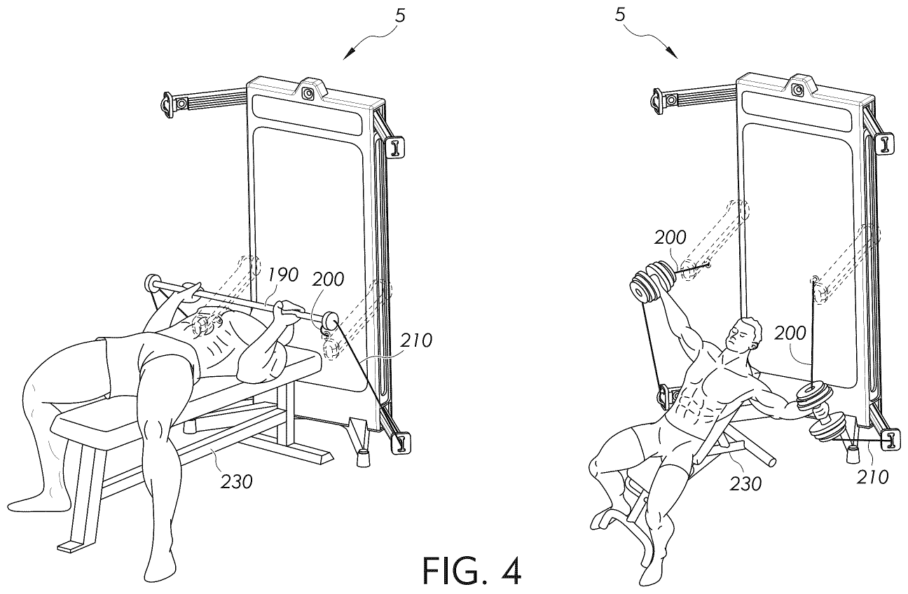

FIG. 4 is a diagram illustrating a side view of a bench accessory (230) that aids in creating additional exercises that require back support or body positioning that cannot be achieved from a standing position. The drawing depicts two examples of how the bench can be positioned in a flat position or in an incline position but does not limit the number of potential accessories to only bench configurations.

The first image depicts a male user activating his pectoral muscles by pressing up an accessory bar (190) with multiple attachment zones on the bars outer edge (210) and (220) that are attached to the both the middle arm cable and the bottom cable applying multiple intensity on the body during the exercise. The second image depicts a male user activating his pectoral muscles by pressing up two dumbbells (220) with multiple attachment zones on the dumbbells outer edge (210) and (220) that are attached to the both the middle arm cable and the bottom cable applying multiple intensity on the body during the exercise.

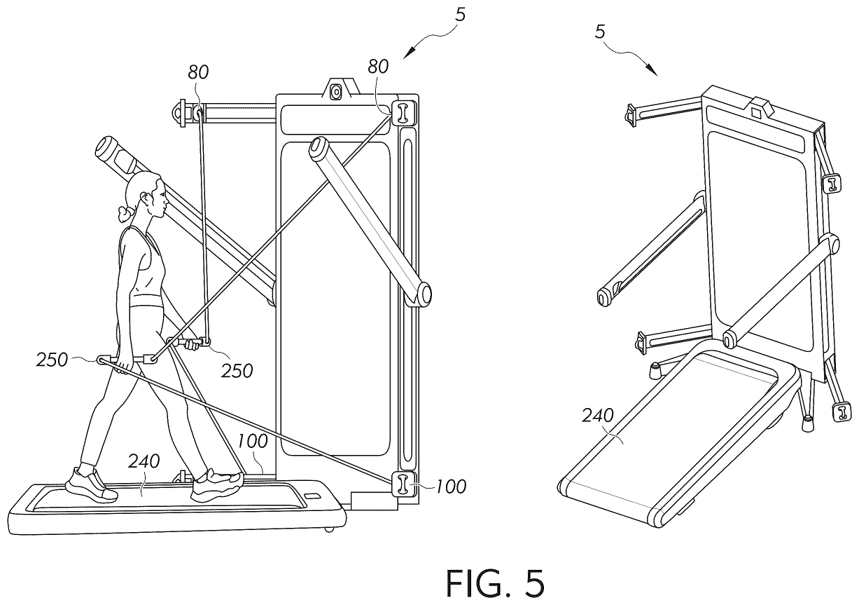

FIG. 5 is a diagram illustrating a side view of a treadmill accessory (240) that aids in creating cardio-based exercises when coupled with the machine. The female user is walking on a treadmill while performing a series of upper arm exercises with handle accessory (250) connected to both the bottom cable (100) and the upper cable (80) applying directional intensity to the body during exercise. The first image depicts the female user walking on a treadmill at 0% grade while the second image illustrates how the treadmill may be inclined to increase the intensity of the exercise.

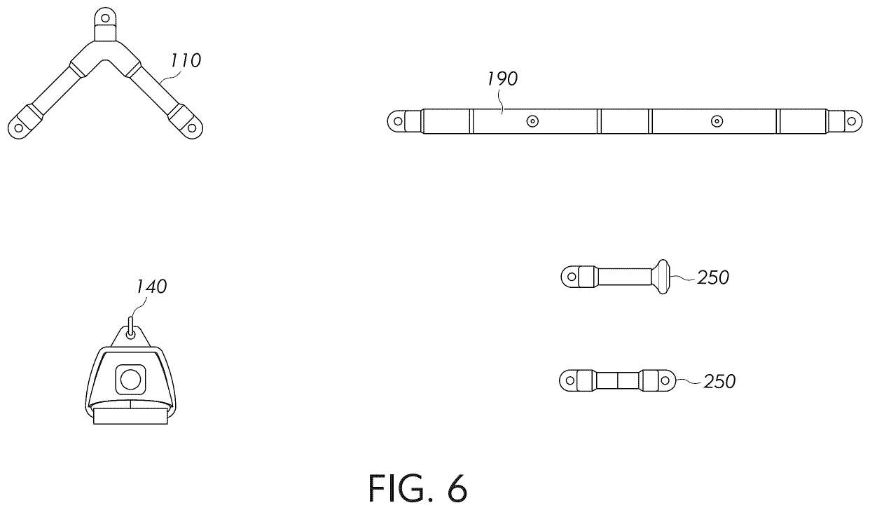

FIG. 6 is a diagram illustrating four possible accessories that link the human body to the invention: 1) triceps bar (110), 2) multiple directional handles (140), 3) long bar (190), and 4) swivel handles (250). Each of these accessories have multiple attachment zones that apply directional intensity to the body. Additional accessories may take the form of additional bars, vests, belts, straps or handles or any other method of linking the body to the invention with multiple attachment zones.

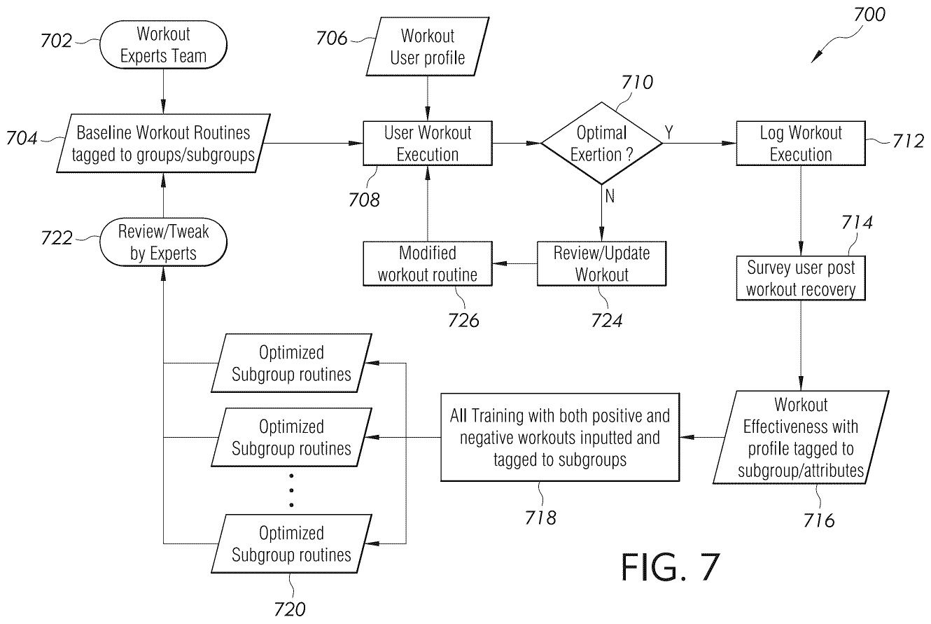

FIG. 7 depicts a flow chart 700 on how the machine learning or artificial intelligence of the invention takes information from the workout creation team and couples it with real feedback from users to modify and perfect workouts for each user across user groups and sub-groups.

The flow may start with a workout experts team 702 generating a baseline workout routine 704 for groups or subgroups. One or more users within the groups or subgroups may execute the workout 708. Additionally, in an example, a workout user profile 706 may be input, e.g., prior to the groups or subgroups executing the workout 708. A determination may be made if execution was “optimal,” 710 e.g., in some example embodiments, optimal may be “group or sub-group” acceptance where no member of the group was removed from the group based on negative feedback. In other embodiments, optimal may be “group or sub-group” acceptance where no more than 1%, 5%, 10%, or some other predetermined percentage of the members of the group are removed from the group based on negative feedback. Optimal may include one or more human determinations, e.g., positive feedback or negative feedback. These human determinations may be input to a system implementing the systems and methods described herein. The determinations may be further processed to determine a percentage of negative feedback below (or above) some predetermined threshold, e.g., 1%, 5%, 10%, or some other predetermined percentage.

Optimal workouts may be logged 712 and a survey of post workout recovery may be performed 714. Workout effectiveness 716 may be determined and AI training 718 with positive and/or negative workouts inputted and tagged may be performed. Optimal subgroup routines 720 may be determined and reviewed and modified 722 by the experts. When exertion if not optimal (710) the workout may be reviewed and/or updated 724 and a modified workout routine 726 may be added to the user workout execution 708.

An example aspect may include an exercise machine.

An example aspect may include a multiple directional force component configured to apply a multiple directional force to an exercise machine-human body interface, the multiple directional force component further configured to provide a divergent intensity to the exercise machine-human body interface to provide the human body with a resistance during a physical exercise.

An example aspect may include an AI component configured to control the multiple directional force component.

An example aspect may include an AI component that controls the multiple directional force component based on morphological characteristics and variability of running speed parameters including at least one of stride length, stride frequency, explosive drive force, and arm drive.

An example aspect may include multiple directional force configured to be applied at a safe point of flexion, extension, abduction, circumduction, or rotation.

An example aspect may include multiple directional force configured to be applied at the safe point of flexion, extension, abduction, circumduction, or rotation after an initial flexion joint is in motion.

An example aspect may include divergent intensity that may be a variable intensity. The variable intensity comprising at least one of physical weights, weighted cables, magnetic disc tension, pulley motors, flywheel systems and cables, intensifying band tension, assistance or resistance band tension, continuous variable wave or pulsing forces on the body, or torque tension created by varying angles.

An example aspect may include multiple directional force including a multiple directional intensity with resistance transferred to the body through at least one of handles, bars, gauntlets, straps, belts, vests, or an accessory having multiple attachment zones linking the human body to the machine or system.

An example aspect may include an AI component including a camera configured to perform a virtual body scan.

An example aspect may include multiple directional force components that include at least one attachment zone.

An example aspect may include multiple directional force component includes multiple attachment zones configured to combine to provide the multiple directional force.

An example aspect may include an exercise system, including an exercise machine and at least one of an adjustable bench, a stationary bicycle, or a flat treadmill configured to be utilized to provide for walking, running, riding, or performing other movements while performing other directional intensity training.

An example aspect may include a method of exercise. The method of exercising may include providing a baseline workout routine for a user to a screen of an exercise machine, determining if a user has exerted a desired level of exertion while performing the provided baseline workout routine based on measurements of fitness data measured by at least one of sensors of the exercise machine or external sensors, modifying the baseline workout routine for the user by the exercise machine when the user has not exerted a desired level of exertion while performing the provided baseline workout routine, logging workout execution when the user has exerted a desired level of exertion while performing the provided baseline workout routine, surveying the user after one of the baseline workout routine or a modified baseline workout routine to estimate effectiveness of the baseline workout routine or a modified baseline workout routine, determining workout effectiveness based on the survey and measured body parameters, and updating the baseline workout routine based on the workout effectiveness and any modify baseline workout routine.

An example aspect may include updating the baseline workout routine. Updating the baseline workout routine may include optimizing a series of subgroup routines.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

1. An exercise machine, comprising:
a multiple directional force component configured to apply a multiple directional force to an exercise machine-human body interface, the multiple directional force component further configured to provide a divergent intensity to the exercise machine-human body interface to provide the human body with a resistance during a physical exercise; and
an artificial intelligence (AI) component configured to control the multiple directional force component.

2. The exercise machine of claim 1, wherein the AI component controls the multiple directional force component based on morphological characteristics and variability of running speed parameters including at least one of stride length, stride frequency, explosive drive force, and arm drive.

3. The exercise machine of claim 1, wherein the multiple directional force is configured to be applied at a safe point of flexion, extension, abduction, circumduction, or rotation.

4. The exercise machine of claim 3, wherein the multiple directional force is configured to be applied at the safe point of flexion, extension, abduction, circumduction, or rotation after an initial flexion joint is in motion.

5. The exercise machine of claim 1, wherein the divergent intensity comprises a variable intensity, the variable intensity comprising at least one of physical weights, weighted cables, magnetic disc tension, pulley motors, flywheel systems and cables, intensifying band tension, assistance or resistance band tension, continuous variable wave or pulsing forces on the body, or torque tension created by varying angles.

6. The exercise machine of claim 1, wherein the multiple directional force comprises a multiple directional intensity with resistance transferred to the body through at least one of handles, bars, gauntlets, straps, belts, vests, or an accessory having multiple attachment zones linking the human body to the machine or system.

7. The exercise machine of claim 1, wherein the AI component further comprises a camera configured to perform a virtual body scan.

8. The exercise machine of claim 1, wherein the multiple directional force component includes at least one attachment zone.

9. The exercise machine of claim 1, wherein the multiple directional force component includes multiple attachment zones configured to combine to provide the multiple directional force.

10. An exercise system, comprising:
an exercise machine including:
a multiple directional force component configured to apply a multiple directional force to an exercise machine-human body interface, the multiple directional force component further configured to provide a divergent intensity to the exercise machine-human body interface to provide the human body with a resistance during a physical exercise, and
an artificial intelligence (AI) component configured to control the multiple directional force component; and
at least one of an adjustable bench, a stationary bicycle, or a flat treadmill configured to be utilized to provide for walking, running, riding, or performing other movements while performing other directional intensity training.

11. The exercise system of claim 10, wherein the AI component controls the multiple directional force component based on morphological characteristics and variability of running speed parameters including at least one of stride length, stride frequency, explosive drive force, and arm drive.

12. The exercise system of claim 10, wherein the multiple directional force is configured to be applied at a safe point of flexion, extension, abduction, circumduction, or rotation.

13. The exercise system of claim 12, wherein the multiple directional force is configured to be applied at the safe point of flexion, extension, abduction, circumduction, or rotation after an initial flexion joint is in motion.

14. The exercise system of claim 10, wherein the divergent intensity comprises a variable intensity, the variable intensity comprising at least one of physical weights, weighted cables, magnetic disc tension, pulley motors, flywheel systems and cables, intensifying band tension, assistance or resistance band tension, continuous variable wave or pulsing forces on the body, or torque tension created by varying angles.

15. The exercise system of claim 10, wherein the multiple directional force comprises a multiple directional intensity with resistance transferred to the body through at least one of handles, bars, gauntlets, straps, belts, vests, or an accessory having multiple attachment zones linking the human body to the machine or system.

16. The exercise system of claim 10, wherein the AI component further comprises a camera configured to perform a virtual body scan.

17. The exercise system of claim 10, wherein the multiple directional force component includes at least one attachment zone.

18. The exercise system of claim 10, wherein the multiple directional force component includes multiple attachment zones configured to combine to provide the multiple directional force.

19. A method of exercise, comprising
providing a baseline workout routine for a user to a screen of an exercise machine;
determining if a user has exerted a desired level of exertion while performing the provided baseline workout routine based on measurements of fitness data measured by at least one of sensors of the exercise machine or external sensors;
modifying the baseline workout routine for the user by the exercise machine when the user has not exerted a desired level of exertion while performing the provided baseline workout routine;
logging workout execution when the user has exerted a desired level of exertion while performing the provided baseline workout routine;
surveying the user after one of the baseline workout routine or a modified baseline workout routine to estimate effectiveness of the baseline workout routine or a modified baseline workout routine;
determining workout effectiveness based on the survey and measured body parameters; and
updating the baseline workout routine based on the workout effectiveness and any modify baseline workout routine.

20. The method of claim 19, wherein updating the baseline workout routine comprises optimizing a series of subgroup routines.
